(19) United States
(12) Patent Application Publication
(10) Pub. No.: US 2024/0165519 A1
(43) Pub. Date: May 23, 2024

(54) PULSE WAVE DETECTION DEVICE, PULSE WAVE DETECTION METHOD, AND PULSE WAVE DETECTION PROGRAM

(71) Applicant: OMRON CORPORATION, Kyoto-shi, Kyoto (JP)

(72) Inventors: Chao ZHANG, Kyoto-shi (JP); Masahiko OGAWA, Kyoto-shi (JP); Hiroyuki IBUKI, Kyoto-shi (JP)

(73) Assignee: OMRON CORPORATION, Kyoto-shi, Kyoto (JP)

(21) Appl. No.: 18/551,197

(22) PCT Filed: Mar. 18, 2022
(86) PCT No.: PCT/JP2022/012670

§ 371 Date: Sep. 19, 2023

(30) Foreign Application Priority Data
Apr. 27, 2021 (JP) 2021-075160

(51) Int. Cl. A63F 13/79; A63B 24/00; A63F 13/213; A63F 13/537; A63F 13/86

(52) U.S. Cl. CPC A63F 13/79; A63B 24/0062; A63F 13/213; A63F 13/537; A63F 13/86; A63B 2230/04

(57) ABSTRACT

A content evaluation device is a pulse wave detection device that detects the pulse wave of a player during game play, comprising an image acquisition unit, a detection area setting unit, and a pulse wave detection unit. The image acquisition unit acquires an image including a player. The detection area setting unit sets a detection area that includes the skin of the player included in the image acquired by the image acquisition unit. The pulse wave detection unit detects the pulse wave of the player from the detection area set in the detection area setting unit.

### TECHNICAL FIELD

The present invention relates to a pulse wave detection device, a pulse wave detection method, and a pulse wave detection program for detecting pulse waves of game players and viewers, for example.

### BACKGROUND ART

In recent years, heart rate, stress level, vascular age, etc., have been evaluated using pulse waves, in which a change in the volume of blood vessels that occur as the heart pumps out blood is detected as a waveform, and this is a type of human biometric information.

For example, Patent Literature 1 discloses a pulse wave detection device, and a content evaluation system comprising the same, in which a frequency band component from which a pulse wave can be taken is extracted from a luminance signal representing each pixel included in a biological region extracted from an image of a living body captured by a camera, a pulse wave is detected from the luminance signal of the biological region from which the frequency band component was extracted, luminance information related to the display of a specific screen is acquired, and the luminance information related to the screen display is used to control whether or not to execute pulse wave detection.

Also, Patent Literature 2 discloses a program for determining whether a user is in a sleeping state by detecting the pulse, heartbeat, heart sound, blood pressure, pulse wave, breathing sound, electrocardiographic potential, skin temperature, amount of perspiration, voiceprint, or other such biometric information in a game that proceeds during sleep.

### CITATION LIST

### Patent Literature

Patent Literature 1: JP-A 2014-221172

Patent Literature 2: JP-A 2020-151597

### SUMMARY

### Technical Problem

However, the following problems are encountered with the conventional system and program described above.

That is, with the content evaluation system disclosed in Patent Literature 1, the content is evaluated using the detection result for the pulse wave associated with a content image, but this does not really take into account content in which there are severe fluctuations in the pulse wave, such as with a player who is playing a game.

Also, with the program disclosed in Patent Literature 2, biometric information such as the pulse of a user playing a game is detected to determine whether or not the user is in a sleeping state, but no consideration is given to estimating the level of concentration, excitement, etc., based on of pulse wave fluctuations in the player during game play.

It is an object of the present invention to provide a pulse wave detection device, a pulse wave detection method, and a pulse wave detection program with which the state of the player with respect to a game, etc., can be estimated by detecting, in a non-contact manner, the pulse wave of the player while playing the game, etc.

### Solution to Problem

The pulse wave detection device according to the first invention is a pulse wave detection device for detecting a pulse wave of a player playing a game, comprising an image acquisition unit, a detection area setting unit, and a pulse wave detection unit. The image acquisition unit acquires an image including the player. The detection area setting unit sets a detection area including the skin of the player included in the image acquired by the image acquisition unit. The pulse wave detection section detects the player's pulse wave from the detection area set in the detection area setting unit.

Here, an image of the player playing the game is acquired, the face or another part included in the acquired image is set as the detection area, and the pulse wave of the player is detected.

Here, the acquired image of the game player may be, for example, an image of the player displayed in the game video, or may be an image captured by a camera or the like separately from the game video. Also, the acquired image may be a moving picture such as a video, or may be a still image that is continuously captured.

A pulse wave is a kind of human biometric information, for example, and refers to a waveform indicating a change in the volume of blood vessels that occurs as the heart pumps out blood. A pulse wave can be detected, for example, by a reflective pulse wave sensor that detects the reflection of infrared rays or the like emitted toward the living body, or by a transmissive pulse wave sensor that detects a change in the blood flow, which varies with the pumping of the heart, when infrared rays or the like are emitted from the surface of the human body, as the amount of change in light passing through the body.

A detection area for detecting a pulse wave is set to a portion of the skin of the person included in an image, such as a person's face.

Consequently, the player's pulse wave can be detected in a non-contact manner, without the player having to wear a device for pulse wave detection.

This allows the state of the player with respect to the game to be estimated by detecting the pulse wave of the player playing the game in a non-contact manner.

The pulse wave detection device according to the second invention is the pulse wave detection device according to the first invention, further comprising a concentration estimation unit that estimates the level of concentration of the player with respect to the game according to the detection result of the pulse wave of the player.

Consequently, whether or not the game is interesting to the player, for example, can be evaluated by using the player's level of concentration on the game, which is estimated using the pulse wave that varies with heart rate and the degree of stress, as an index.

The pulse wave detection device according to the third invention is the pulse wave detection device according to the second invention, further comprising an evaluation unit that evaluates the game on the basis of the estimation result of the concentration estimation unit.

Consequently, the game can be evaluated more accurately by evaluating whether or not the game is interesting by using the player's level of concentration on the game, which is estimated using pulse waves that vary with the heart rate and the degree of stress, as an index.

The pulse wave detection device according to the fourth invention is the pulse wave detection device according to the third invention, wherein the evaluation unit evaluates the game according to the estimation result for the level of concentration of a plurality of the players.

Consequently, the game can be evaluated more accurately, without relying on individual differences, by using the result of estimating the level of concentration of a plurality of players playing the same game.

The pulse wave detection device according to the fifth invention is the pulse wave detection device according to the third or fourth invention, wherein the evaluation unit evaluates the game when a state in which the level of concentration by the player is at or over a specific threshold has lasted for at least a specific number of times or for at least a specific length of time.

Consequently, the game can be evaluated more accurately by evaluating the game according to the number of times, or how long, the player's level of concentration on the game has been at or over a specific threshold when the player's estimated level of concentration on the game is quantified, for example.

The pulse wave detection device according to the sixth invention is the pulse wave detection device according to any of the second to fifth inventions, further comprising a display control unit that causes the estimation result of the concentration estimation unit to be displayed on the game display screen.

Consequently, how much a player is currently concentrating can be easily recognized by displaying the estimation result for the player's concentration level on the screen of the game during play.

The pulse wave detection device according to the seventh invention is the pulse wave detection device according to any of the second to sixth inventions, further comprising an intense scene detection unit that detects an exciting scene of the game on the basis of the level of concentration of a plurality of the players with respect to the game, estimated by the concentration estimation unit according to the detection result of the pulse wave detection unit.

Consequently, the result of estimating the level of concentration on the game of a plurality of players playing the game can be used as an index to detect an exciting scene in the game, such as a battle scene.

The pulse wave detection device according to the eighth invention is the pulse wave detection device according to the seventh invention, further comprising an intense scene prediction unit that predicts the intense scene on the basis of the estimation result of the concentration estimation unit.

Consequently, a viewer who is watching a game video, for example, can watch an intense scene that is expected to occur afterward, without missing it, by predicting that scene according to the estimation result for the level of concentration estimated according to the fluctuation in the pulse wave of the player playing the game.

The pulse wave detection device according to the ninth aspect of the invention is the pulse wave detection device according to the eighth aspect of the invention, further comprising a communication control unit that performs push communication to a viewer of the game on the basis of the basis of the prediction result of the intense scene prediction unit.

Consequently, a viewer can be prompted not to miss an intense scene by notifying the viewer of the game of the prediction of the occurrence of an intense scene, which has been predicted on the basis of the estimation result of the level of concentration estimated according to the fluctuation in the pulse wave, etc.

The pulse wave detection device according to the tenth invention is the pulse wave detection device according to any of the second to ninth inventions, wherein the concentration estimation unit estimates the level of concentration for each player by taking into account the difficulty level of the game and the ability of each player.

Here, in estimating the level of concentration of a player of a game, it is important to consider whether the difficulty of the game and the ability of the player are in a balanced state (flow state).

For instance, if the degree of difficulty of the game is too high for the player's ability, it will be difficult for the player to clear the game, so we can expect the level of concentration not to increase. Conversely, if the degree of difficulty of the game is too low for the player's ability, it will be easy for the player to clear the game, so once again we can expect the level of concentration not to increase.

Consequently, in estimating the level of concentration of a player in a game, whether or not the difficulty of the game is well matched to the ability of the player can be used as a new index, in addition to the pulse wave, to estimate the level of concentration of each player, and this allows the game to be evaluated more accurately.

The pulse wave detection device according to the eleventh invention is the pulse wave detection device according to any of the second to tenth inventions, wherein the image acquisition unit acquires an image of a viewer who is viewing the game screen, and the concentration estimation unit uses an image of the viewer to estimate the level of concentration of the viewer.

Consequently, the game can be evaluated more accurately, according to the viewer's level of concentration, by using an image of the viewer watching the video of the game being played to estimate the viewer's level of concentration on the game.

The pulse wave detection device according to the twelfth invention is the pulse wave detection device according to the eleventh invention, further comprising a gift control unit that controls execution of a gift function from the viewer to a distributor who is distributing the video of the game, on the basis of the estimation result for the concentration level of the viewer estimated by the concentration estimation unit.

Consequently, when it is detected that the game is becoming more exciting on the basis of the level of concentration of the viewer watching the game screen, for example, control can be performed to execute the gift function for the distributor who is distributing the video of that game.

The pulse wave detection device according to the thirteenth invention is the pulse wave detection device according to the eleventh or twelfth invention, further comprising an evaluation unit that evaluates the game on the basis of the estimation result for the concentration estimation unit. The evaluation unit evaluates the game on the basis of the level of concentration of both the player and the viewer.

Consequently, the game can be evaluated more accurately, according to the levels of concentration of both the player and the viewer, by using an image not only of the game player, but also a viewer watching the video of the game being played, to estimate the viewer's level of concentration on the game.

The pulse wave detection device according to the fourteenth invention is the pulse wave detection device according to any of the eleventh to thirteenth inventions, further comprising a communication control unit that performs push communication to the viewer of the game on the basis of the estimation result for the concentration level of the viewer estimated by the concentration estimation unit.

Consequently, it can be detected that the game is becoming more exciting according to the estimated level of concentration of the viewer, and viewers of other games can be prompted to view this game by notifying those viewers of an exciting game scene according to the degree of excitement.

The pulse wave detection device according to the fifteenth invention is the pulse wave detection device according to any of the first to fourteenth inventions, further comprising an emotion estimation unit that estimates the player's emotion on the basis of information about the facial portion of the player set in the detection area setting unit.

Consequently, the game can be evaluated more accurately by including concentration, joy, disappointment, and other such emotions estimated from the player's facial expression and so forth, in addition to the pulse wave, as an index for evaluating the game.

The pulse wave detection method according to the sixteenth invention is a pulse wave detection method for detecting a pulse wave of a player while playing a game, the method comprising an image acquisition step, a detection area setting step, and a pulse wave detection step. In the image acquisition step, an image including the player is acquired. In the detection area setting step, a detection area is set that includes the skin of the player included in the image acquired in the image acquisition step. In the pulse wave detection step, the player's pulse wave is detected from the detection area set in the detection area setting step.

Consequently, the same effect can be obtained as that obtained with the pulse wave detection device according to the first invention.

The pulse wave detection program according to the seventeenth invention causes a computer to execute a pulse wave detection method for detecting a pulse wave of a player playing a game, the method comprising an image acquisition step, a detection area setting step, and a pulse wave detection step. In the image acquisition step, an image including the player is acquired. In the detection area setting step, a detection area is set that includes the skin of the player included in the image acquired in the image acquisition step. In the pulse wave detection step, the player's pulse wave is detected from the detection area set in the detection area setting step.

Consequently, the same effect can be obtained as that obtained with the pulse wave detection device according to the first invention.

The pulse wave detection device according to the eighteenth invention comprises an image acquisition unit, a detection area setting unit, and a pulse wave detection unit. The image acquisition unit acquires an image including a viewer who is watching a game screen during game play. The detection area setting unit sets a detection area including the skin of the viewer included in the image acquired by the image acquisition unit. The pulse wave detection unit detects the pulse wave of the viewer from the detection area set in the detection area setting unit.

Here, an image of the viewer looking at the screen of the game being played by the player is acquired, the face or another such part of the viewer included in the acquired image is set as the detection area, and the pulse wave of the viewer looking at the game screen is detected.

Here, the acquired image of the viewer of the game screen may be, for example, an image of the viewer that is displayed in the game video, or may be an image captured by a camera or the like separately from the game screen. Also, the acquired image may be a moving picture such as a video, or may be still images that are continuously captured.

A pulse wave is a kind of human biometric information, for example, and refers to a waveform that indicates a change in the volume of blood vessels that occurs as the heart pumps out blood. A pulse wave can be detected, for example, by a reflective pulse wave sensor that detects the reflection of infrared rays or the like emitted toward the living body, or by a transmissive pulse wave sensor that detects a change in the blood flow, which varies with the pumping of the heart, when infrared rays or the like are emitted from the surface of the human body, as the amount of change in light passing through the body.

A detection area for detecting a pulse wave is set to a portion of the skin of the person included in an image, such as a person's face.

Consequently, the viewer's pulse wave can be detected in a non-contact manner, without the viewer who is watching the game having to wear a device for pulse wave detection.

Therefore, the pulse wave of the viewer watching the screen of the game being played can be detected without contact, and the state of the viewer can be estimated.

A pulse wave detection device according to the nineteenth invention is the pulse wave detection device according to the eighteenth invention, further comprising a concentration estimation unit that estimates the level of concentration on the part of the viewer with respect to the game according to the detection result of the pulse wave of the viewer.

Consequently, whether or not the game is interesting to the viewer, for example, can be evaluated by using the viewer's level of concentration on the game, which is estimated using the pulse wave that varies with heart rate and the degree of stress, as an index.

The pulse wave detection device according to the twentieth aspect of the invention is the pulse wave detection device according to the nineteenth invention, further comprising an evaluation unit that evaluates the game on the basis of the estimation result of the concentration estimation unit.

Consequently, the game can be evaluated more accurately by evaluating whether or not the game is interesting by using the viewer's level of concentration on the game, which is estimated using pulse waves that vary with the heart rate and the degree of stress, as an index.

The pulse wave detection device according to the twenty-first invention is the pulse wave detection device according to the twentieth invention, wherein the evaluation unit evaluates the game according to the estimation result for the level of concentration of a plurality of the viewers.

Consequently, the game can be evaluated more accurately, without relying on individual differences, by using the result of estimating the level of concentration of a plurality of viewers watching the same game.

The pulse wave detection device according to the twenty-second invention is the pulse wave detection device according to the twentieth or twenty-first invention, wherein the evaluation unit evaluates the game when a state in which the level of concentration by the viewer is at or over a specific threshold has lasted for at least a specific number of times or for at least a specific length of time.

Consequently, the game can be evaluated more accurately by evaluating the game according to the number of times, or how long, the viewer's level of concentration on the game has been at or over a specific threshold when the viewer's estimated level of concentration on the game is quantified, for example.

The pulse wave detection device according to the twenty-third invention is the pulse wave detection device according to any of the nineteenth to twenty-second inventions, further comprising a gift control unit that controls execution of a gift function from the viewer to a distributor who is distributing the video of the game, on the basis of the estimation result for the concentration level of the viewer estimated by the concentration estimation unit.

Consequently, when it is detected that the game is becoming more exciting on the basis of the level of concentration of the viewer watching the game screen, for example, control can be performed to execute the gift function for the distributor who is distributing the video of that game.

The pulse wave detection device according to the twenty-fourth invention is the pulse wave detection device according to any of the nineteenth to twenty-third inventions, further comprising a communication control unit that performs push communication to the viewer of the game on the basis of the estimation result for the concentration level of the viewer estimated by the concentration estimation unit.

Consequently, a viewer can be prompted not to miss an intense scene by notifying the viewer of the game of the prediction of the occurrence of an intense scene, which has been predicted on the basis of pulse wave fluctuations, etc.

The pulse wave detection device according to the twenty-fifth invention is the pulse wave detection device according to any of the nineteenth to twenty-fourth inventions, further comprising an intense scene detection unit that detects an exciting scene of the game on the basis of the level of concentration of a plurality of the viewers with respect to the game, estimated by the concentration estimation unit according to the detection result of the pulse wave detection unit.

Consequently, the result of estimating the level of concentration on the game of a plurality of viewers watching the screen of the game being played can be used as an index to detect an exciting scene in the game, such as a battle scene.

The pulse wave detection device according to the twenty-sixth invention is the pulse wave detection device according to the twenty-fifth invention, further comprising an intense scene prediction unit that predicts the intense scene from the estimation result of the concentration estimation unit.

Consequently, an intense scene that is expected to occur later can be predicted on the basis of the estimation result for the level of concentration estimated according to the fluctuation in the pulse waves of a viewer of the screen of the game being played, allowing other viewers to view the scene, without missing it.

The pulse wave detection device according to the twenty-seventh aspect of the present invention is the pulse wave detection device according to the twenty-sixth aspect, further comprising a communication control unit that performs push communication to a viewer of the game on the basis of the prediction result of the intense scene prediction unit.

Consequently, other viewers can be prompted not to miss an intense scene by notifying those viewers of the prediction of the occurrence of an intense scene predicted on the basis of the estimation result of the level of concentration estimated according to the fluctuation in the viewer's pulse wave, etc.

The pulse wave detection device according to the twenty-eighth invention is the pulse wave detection device according to any of the nineteenth to twenty-seventh inventions, wherein the image acquisition unit acquires an image of a player playing the game, and the concentration estimation unit uses an image of the player to estimate the level of concentration of the player.

Consequently, the game can be evaluated more accurately, according to the level of concentration of the player, by using an image of the player playing the game to estimate the level of concentration of the player on the game.

The pulse wave detection device according to the twenty-ninth invention is the pulse wave detection device according to the twenty-eighth invention, further comprising an evaluation unit that evaluates the game on the basis of the estimation result of the concentration estimation unit. The evaluation unit evaluates the game on the basis of the level of concentration of both the viewer and the player.

Consequently, the game can be evaluated more accurately, according to the levels of concentration of both the player and the viewer, by using an image not only of the viewer of the screen during game play, but also of the player who is actually playing the game, to estimate the viewer's level of concentration on the game.

The pulse wave detection device according to the thirtieth invention is the pulse wave detection device according to any of the eighteenth to twenty-ninth inventions, further comprising an emotion estimation unit that estimates the viewer's emotion on the basis of information about the facial portion of the viewer set in the detection area setting unit.

Consequently, the game can be evaluated more accurately by including concentration, joy, disappointment, and other such emotions estimated from the facial expression and so forth of a game screen viewer, in addition to the pulse wave, as an index for evaluating the game.

The pulse wave detection method according to the thirty-first invention comprises an image acquisition step, a detection area setting step, and a pulse wave detection step. In the image acquisition step, an image including a viewer who is watching a game screen during game play is acquired. In the detection area setting step, a detection area including the skin of the viewer included in the image acquired in the image acquisition step is set. In the pulse wave detecting step, the pulse wave of the viewer is detected from the detection area set in the detection area setting step.

Consequently, the same effect can be obtained as that obtained with the pulse wave detection device according to the eighteenth invention.

The pulse wave detection program according to the thirty-second invention causes a computer to execute a pulse wave detection method, the method comprising an image acquisition step, a detection area setting step, and a pulse wave detection step. In the image acquisition step, an image including the a viewer who is watching a game screen during game play is acquired. In the detection area setting step, a detection area is set that includes the skin of the player included in the image acquired in the image acquisition step. In the pulse wave detection step, the viewer's pulse wave is detected from the detection area set in the detection area setting step.

Consequently, the same effect can be obtained as that obtained with the pulse wave detection device according to the eighteenth invention.

### Effect

With the pulse wave detection device according to the present invention, the state of a player or the like with respect to a game can be estimated by detecting the pulse wave of the player who is playing the game, etc., in a non-contact manner.

### BRIEF DESCRIPTION OF THE DRAWINGS

FIG. 1 is a control block diagram of the configuration of a content evaluation system including a content evaluation device according to an embodiment of the present invention;

FIG. 2 is a schematic diagram of a situation in which two players in a battle game and a large number of viewers are connected over an Internet line;

FIG. 3 shows map of the level of concentration of the player playing a game;

FIG. 4 shows an example of the screen during play of the game to be evaluated;

FIG. 5 is a graph of the relation between the elapsed time up to an intense scene in the game and changes in the level of concentration of the player, which illustrates the prediction of an intense scene;

FIG. 6 is a diagram of an example of services for viewers, distributors, etc., using images captured by cameras installed on the player side and the viewer side;

FIG. 7 is a main flowchart showing the flow of processing in a content evaluation method carried out by the content evaluation device of FIG. 1;

FIG. 8 is a flowchart showing the flow of processing for detecting and displaying the intensity and occurrence frequency based on the player's level of concentration in the game, as a specific example of the control in step S17 of FIG. 7;

FIG. 9 is a flowchart showing the flow of processing for searching for intense scenes that people will want to view based on the level of concentration of the player on the game, as a specific example of the control in step S17 of FIG. 7;

FIG. 10 is a flowchart showing the flow of processing for performing push communication to a viewer according to a prediction of intensity tendency based on the level of concentration of the player on the game, as a specific example of control in step S17 of FIG. 7;

FIG. 11 is a flowchart showing the flow of processing for proposing execution of a gift function according to excitement on the viewer side on the basis of the level of concentration of the viewer, as a specific example of control in step S17 of FIG. 7; and

FIG. 12 is a flowchart showing the flow of processing for performing push communication to other viewers according to the statistics for excitement on the viewer side on the basis of the level of concentration of a viewer, as a specific example of the control in step S17 of FIG. 7.

### DESCRIPTION OF EMBODIMENTS

### Embodiment 1

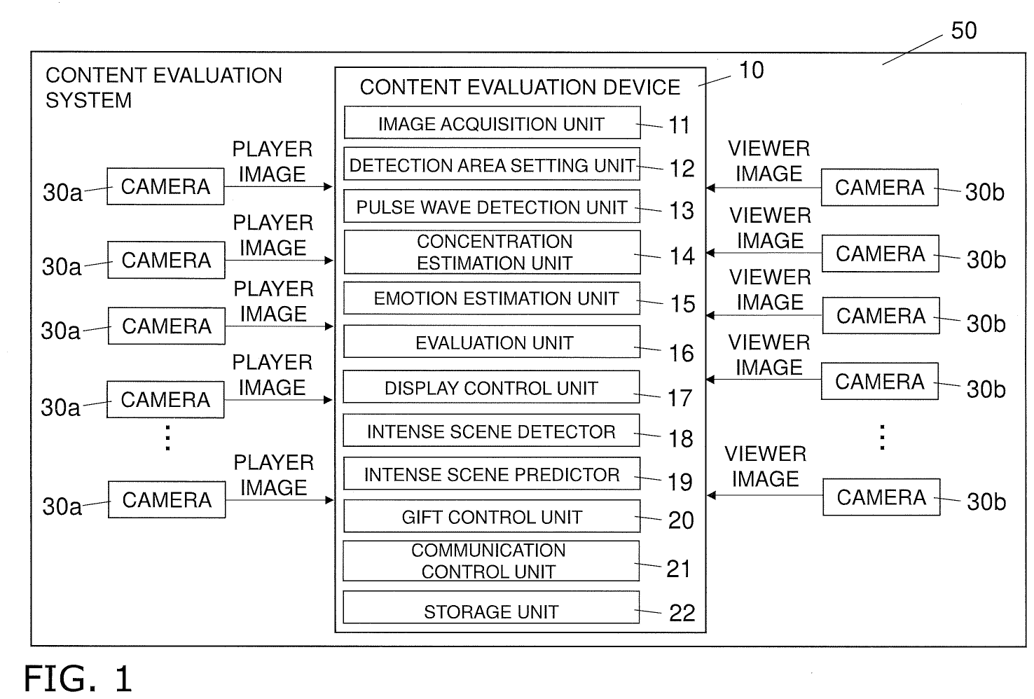

A content evaluation system 50 including a content evaluation device (pulse wave detection device) 10 according to an embodiment of the present invention will now be described with reference to FIGS. 1 to 12.

The content evaluation system 50 of this embodiment is a system for evaluating a game, and as shown in FIG. 1, comprises a plurality of cameras 30a that capture facial images of game players (content user), a plurality of cameras 30b that capture facial images of viewers (content users) of the screen of the game being played, and a content evaluation device 10 to which the facial images of the players and viewers are transmitted from these cameras 30a and 30b.

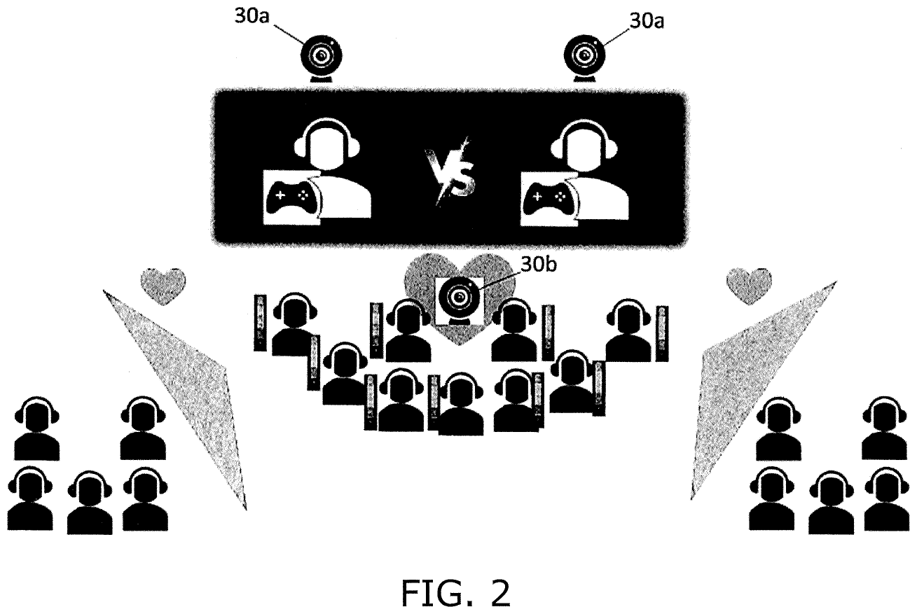

For example, as shown in FIG. 2, the content evaluation system 50 connects two players of a battle game and a large number of viewers over an Internet line, and live-distributes video of the game actual situation. Various services (discussed below) are provided using images captured by cameras 30a installed in front of the two players and cameras 30b installed in front of a large number of viewers. The number of new viewers who are interested in the various services provided in the live-distributed game videos increases so that these viewers can watch the game video.

In FIG. 2, for the sake of description, only one camera 30b installed on the viewer side is shown, but in actual practice a camera 30b is installed in front of each viewer.

With the content evaluation system 50 of this embodiment, in order to evaluate a game, the pulse wave of each person is detected from images of the players of the game being played and the viewers of the game screen, and how interesting the game is (its intensity, etc.) is evaluated according to the level of concentration estimated on the basis of the change in the pulse wave.

The plurality of cameras 30a and 30b are, for example, ordinary cameras equipped with an imaging device such as a CCD (charge coupled device) or a CMOS (complementary metal oxide semiconductor), and convert light detected by the imaging device into an image signal, and output the result.

In this embodiment, the cameras 30a and 30b are, for example, monochrome CCD cameras that capture rectangular images, and output 8-bit (pixel values of 0 to 255) captured image data at a frame rate of 6 frames per second. We shall assume that the number of frames of captured image data outputted in one capture is 64 frames. That is, captured image data obtained by imaging a subject for about 10.7 seconds is outputted to the content evaluation device 10.

The shape, size, number of bits, frame rate, etc., of the images outputted from the cameras 30a and 30b can be changed as needed.

With the content evaluation system 50 of this embodiment, the frame rate of the outputted image is set such that a person's pulse (60 to 80 beats per minute) can be sufficiently captured in order to measure the pulse wave of a person (pulse rate, etc.) as an index for evaluating content such as games.

The cameras 30a and 30b may be infrared cameras rather than monochrome CCD cameras, for example. In this case, the skin part of the facial images of the players and viewers of the game is set as the detection area, and the blood flow in the body can be detected to detect the pulse waves of the players and viewers.

A plurality of cameras 30a are installed in front a plurality of players who are playing a game, capture images including the faces (skin) of the players, and transmit these images to the content evaluation device 10.

A plurality of cameras 30b are installed in front of the plurality of viewers who are looking at the screen of the game being played, capture images including the viewers' faces (skin), and transmit these images to the content evaluation device 10.

Whether or not to display the facial images captured by the cameras 30a and 30b on the game screen 40 can be selected by the players and the viewers. The cameras 30a and 30b may be a built-in camera installed in a PC (personal computer), a web camera connected to a PC, or a camera function installed on a smartphone, tablet terminal, or the like.

The content evaluation device 10 evaluates a game on the basis of information about the players of the game and the viewers of the screen of the game being played. As shown in FIG. 1, the content evaluation device 10 has an image acquisition unit 11, a detection area setting unit 12, a pulse wave detection unit 13, a concentration estimation unit 14, an emotion estimation unit 15, an evaluation unit 16, a display control unit 17, an intense scene detection unit 18, an intense scene prediction unit 19, a gift control unit 20, a communication control unit 21, and a storage unit 22.

The image acquisition unit 11 acquires facial images from the cameras 30a and 30b that capture facial images including the faces of game players and viewers. The image acquisition unit 11 can acquire images from the cameras 30a and 30b wirelessly, such as by Bluetooth (registered trademark), or via a wired communication line.

The detection area setting unit 12 sets, as a detection area, the portion of an image of a game player or viewer acquired by the image acquisition unit 11 in which the person's skin is exposed, such as the face portion.

The pulse wave detection unit 13 detects the pulse waves of players and viewers by performing image processing on the image areas including the faces of the game players and viewers set in the detection area setting unit 12.

The method disclosed in JP-A 2016-159108, for example, can be employed as the means for detecting a pulse wave using an image.

More specifically, for example, specific image processing is performed on a facial image acquired by the image acquisition unit 11 to generate a plurality of sets converted image data in time-series, a feature amount indicating the luminance of the detection area is calculated for each set of converted image data, and pulse information about the players and viewers is generated on the basis of the time-series data of this feature amount. That is, the pulse wave detection unit 13 analyzes the time-series data for the feature amount, and generates information about the pulses of the players and the viewers on the basis of the periodicity of a luminance change.

In this embodiment, the pulse wave detection unit 13 calculates the average luminance of pixels included in the detection area for each of the 64 sets of time-series converted image data, and analyzes the change over time.

Here, the periodic change in the average luminance represents the pulse cycle. Therefore, the pulse wave detection unit 13 finds the fluctuation period of the average luminance. Then, the pulse wave detection unit 13 calculates the pulse rate (beats per minute), which refers to the number of pulses per unit time, from the fluctuation period of the average luminance.

The pulse wave detection unit 13 may generate the amplitude (strength of pulse), the presence or absence of arrhythmia, or the like in addition to the pulse rate as the pulse information.

Also, the method disclosed in JP-A 2017-164215, for example, may be employed as the means for detecting a pulse wave of a person included in a moving picture.

More specifically, the pulse rate of each person can be found on the basis of the change in luminance of their skin in the video (for example, see Xiaobai Li et al., “Remote Heart Rate Measurement From Face Videos Under Realistic Situations,” Computer Vision and Pattern Recognition (CVPR), 2014 IEEE Conference on 23-28 June 2014, pp. 4264-4271).

The concentration estimation unit 14 estimates the level of concentration of the players and the viewers for the game being played on the basis of the results of detecting the pulse waves (pulses, etc.) of the players and the viewers of the game as detected by the pulse wave detection unit 13.

More specifically, the concentration estimation unit 14 estimates the level of concentration of a person by using the pulse wave detection unit 13 to detect the pulse vital data of each person from the facial portions of the players and viewers of the game included in the image acquired by the image acquisition unit 11.

In general, when users (players and viewers) of various content such as games are concentrating, it is empirically known that their pulse rate and body temperature rise, their eyes open wider, they blink less often, and facial movements are reduced.

Therefore, in this embodiment, the concentration estimation unit 14 estimates the level of concentration of each person on the basis of changes in the pulse rates (average value for a specific period of time, variance, etc.) of game players and viewers as detected by the pulse wave detection unit 13.

Consequently, for example, even if a player is playing the game with an expressionless face, the change in the level of concentration behind that expression can be quantified and displayed on the game screen 40, thereby conveying the player's intentions to the viewer.

Also, the concentration estimation unit 14 may use the method disclosed in JP-A 2021-23492 to estimate the level of concentration of each player and viewer of the game, for example.

More specifically, the image acquisition unit 11 acquires image data for a specific number of judgment frames from the cameras 30a and 30b.

Here, the number of judgment frames is a parameter that determines the number of frames, which is used for calculating the level of concentration, and its value is set in advance. For example, with image data of 30 fps, if the number of judgment frames is set to 150, the level of concentration is judged by analyzing the movement (behavior) of a person in a five-second moving picture.

When a person is concentrating on something, their face and gaze tend to move less and their eyes open wider. On the other hand, when attention is distracted or the person becomes drowsy, there is a tendency for the face and gaze to move more and the eyes to open less wide.

In light of the above, in this embodiment, in addition to the subject's pulse detected by the pulse wave detection unit 13, three other factors are also evaluated, namely, “change in face position,” “change in face orientation,” and “eye opening wideness,” and the score obtained by adding up these evaluation points may be defined as the “level of concentration.”

Also, the concentration estimation unit 14 estimates the level of concentration of each player by taking into account the difficulty of the game (such as the strength of the opponent in the case of a battle game) and the ability of the player.

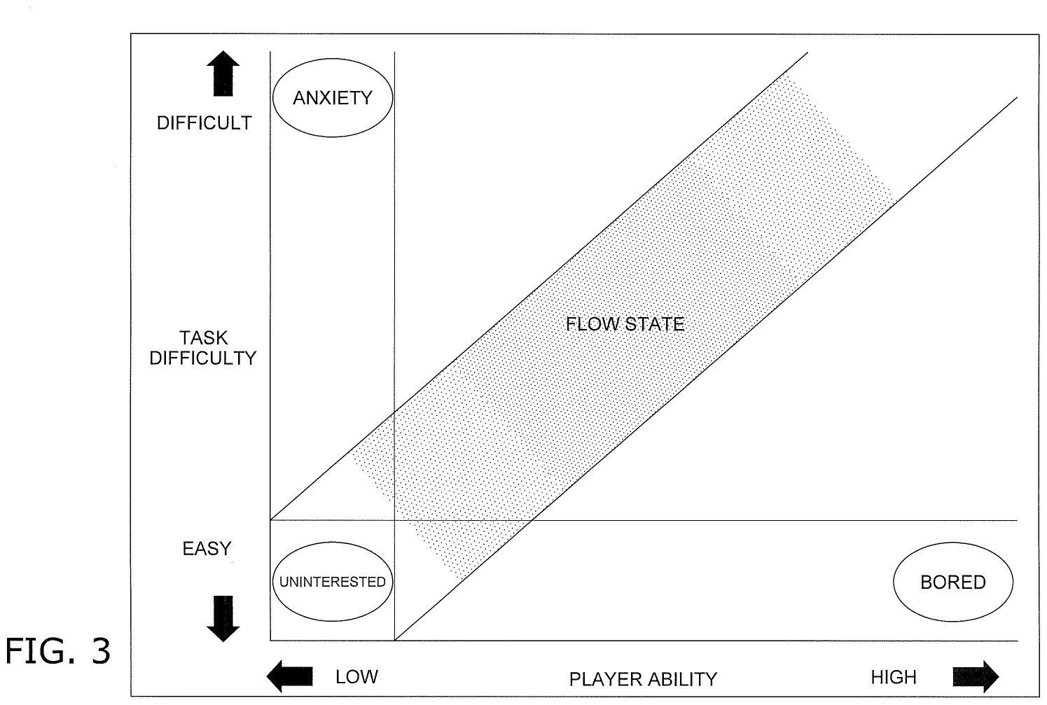

Here, as shown in FIG. 3, it is known that the level of concentration of a person is higher when their own ability and the difficulty of the set task are evenly matched, and the ultimate state of concentration, the so-called “flow state,” is reached.

That is, if the difficulty level of the game is too high for the player's ability, for example, it will be difficult for the player to clear the game, which will cause some anxiety in the player, and there will be no increase in the level of concentration. On the other hand, if the difficulty level of the game is too low for the player's ability, it will be simple for the player to clear the game, and the player will lose interest, so once again there will be no increase in the level of concentration.

Consequently, when the concentration estimation unit 14 estimates the player's level of concentration in the game, whether or not the difficulty of the game and the ability of the player are evenly matched can be used as a new index, in addition to the pulse wave, to estimate more accurately the level of concentration of the player on the game.

In addition to the method discussed above, the estimation of the level of concentration by the concentration estimation unit 14 may be performed, for example, by a method in which a plurality of images are used to calculate a pulse wave/pulse chart within a certain time interval, and the pulse wave peak is extracted to calculate the pulse wave cycle (length), and converting the level of concentration on the basis of the pulse wave variation.

The emotion estimation unit 15 estimates the emotion of each person on the basis of information about the faces of the players and viewers set in the detection area setting unit 12.

More specifically, the emotion estimation unit 15 estimates emotions such as joy, surprise, fear, anger, anger, disgust, and sadness from changes in the facial expressions of game players and viewers included in the images continuously acquired in time series by the image acquisition unit 11.

The estimation result from the emotion estimation unit 15 is used as one of the indices when the evaluation unit 16 evaluates the game.

The method disclosed in JP-A 2016-149063, for example, can be used for estimating human emotions in the emotion estimation unit 15.

The evaluation unit 16 evaluates the degree of intensity, etc., of the game being played on the basis of the estimation result for the level of concentration of the players and the viewers estimated by the concentration estimation unit 14, and changes in the emotions of the players and the viewers.

The evaluation unit 16 does not necessarily have to use the estimation results for the levels of concentration of both the players and the viewers to evaluate the game, and may evaluate the game using the estimation result for just one of these.

Also, the evaluation unit 16 does not necessarily evaluate the game by taking into account the results of estimating the emotions of both the players and the viewers, but the game can be evaluated more accurately if the evaluation includes these emotion estimation results.

The display control unit 17 displays the estimation result from the concentration estimation unit 14 near the player's facial image displayed on the screen of the game, for example.

Consequently, a player who is playing the game can easily recognize an increase or decrease in his or her level of concentration while viewing the game screen.

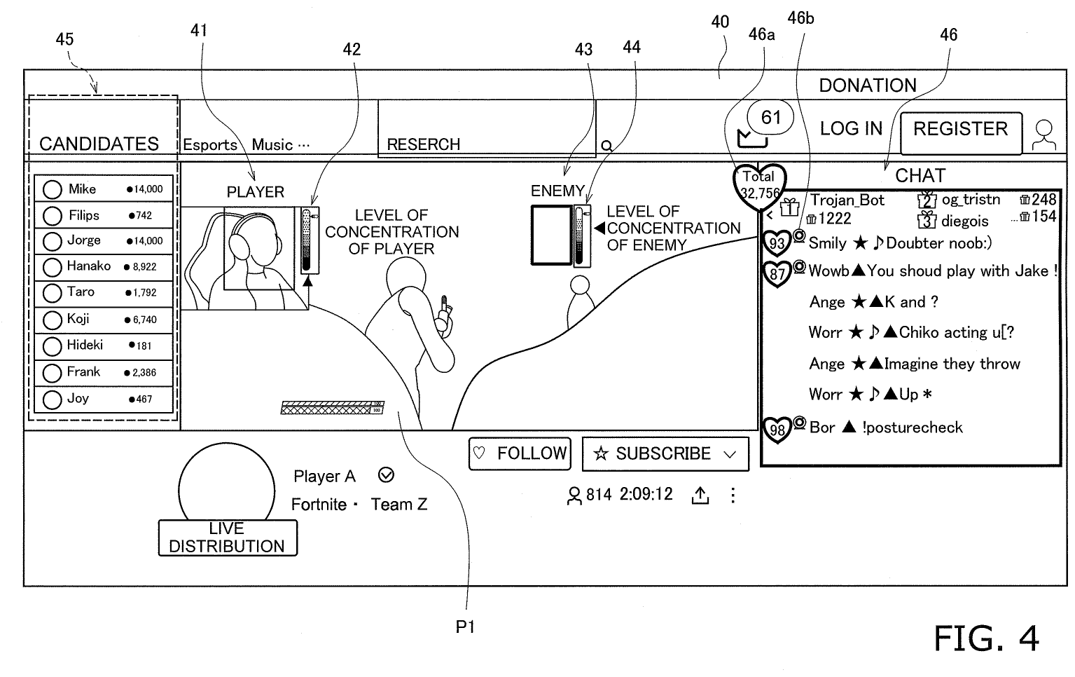

The display control unit 17 also displays the level of concentration of the player and the levels of concentration of other players participating in the game on the game screen 40 shown in FIG. 4, in a barometer-like format, via an Internet line, for example.

This allows the player to recognize not only his or her own level of concentration but also those of other players on the game. Furthermore, viewers watching the game screen can easily recognize the level of concentration of each player participating in the game, so they can select and watch intense scenes where the players are more excited.

The intense scene detection unit 18 detects an exciting scene in the game on the basis of the level of concentration of a plurality of players with respect to the game as estimated by the concentration estimation unit 14 according to the detection result from the pulse wave detection unit 13.

Here, an intense scene is, for example, a scene in which a player is battling another player (enemy), or is battling an enemy player (enemy) controlled by a computer, in the battle-type game shown in FIG. 4, and means an exciting scene in the game as predicted from the level of concentration on the part of a game player.

Such an intense scene is one a viewer watching the game screen will not want to miss, and is the kind of scene that viewers most want to watch.

The intense scene detection unit 18 also detects the frequency with which an intense scene is detected.

Consequently, a viewer can easily recognize the frequency with which intense scenes can be encountered when watching the game, according to the frequency at which intense scenes were detected.

The intense scene prediction unit 19 predicts the occurrence of an intense scene on the basis of the estimation result from the concentration estimation unit 14, as estimated from the changes in the detection result of the pulse wave detection unit 13.

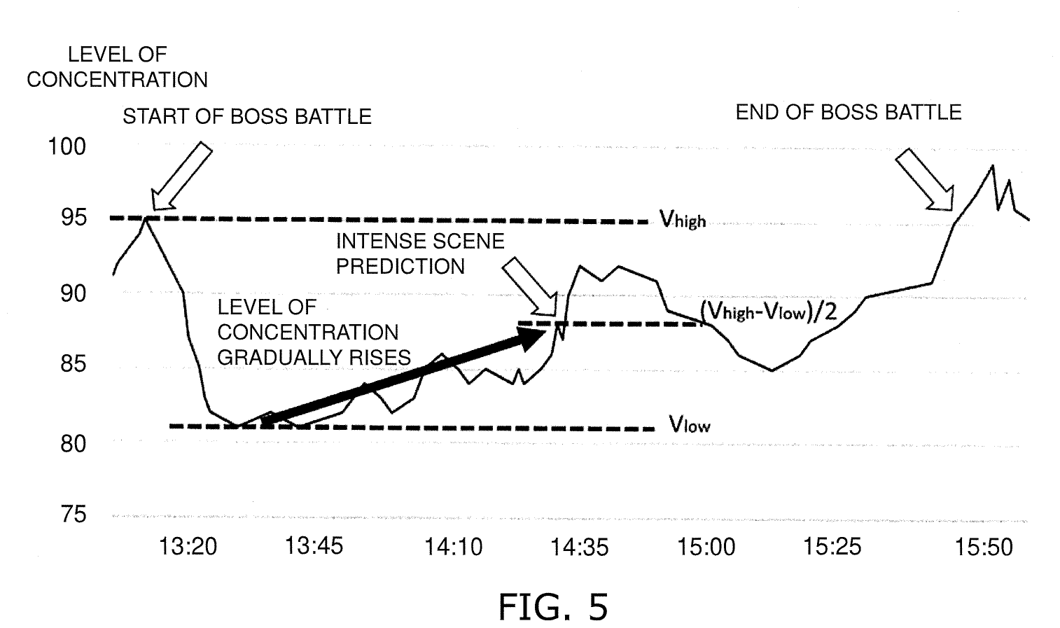

More specifically, as shown in FIG. 5, the intense scene prediction unit 19 predicts whether or not an intense scene will occur on the basis of the changes in the level of concentration of a player on the game as estimated by the concentration estimation unit 14.

In the example shown in FIG. 5, if a battle with a boss character is started in a battle-type game (at around 1:15 p.m.), for example, the level of concentration of the player first drops (at around 1:30 p.m.), but then gradually increases. If we let V_{high} be the highest level of concentration when the battle with the boss character is begun, and let V_{low} be the lowest level of concentration when the level first drops after the start of the game, when the intense scene prediction unit 19 predicts that the player's level of concentration has risen to half that level of concentration (V_{high}−V_{low})/2 (at around 2:30 p.m.), it is predicted that an intense scene will occur in that game after this point. The communication control unit 21 then performs push communication to prompt the viewer to watch the video of the game, for example, on the game screen 40 shown in FIG. 4, over an Internet line.

Consequently, for example, a viewer watching a game can predict the occurrence of an intense scene, and be sure not to miss it, by referring to the prediction result and selecting the screen of the player to watch.

The gift control unit 20 controls a gift function that conveys the viewer's gratitude to the distributor distributing the video of the game, on the basis of the estimation result of the level of concentration of the viewer as estimated by the concentration estimation unit 14.

The distributor may be a player of the game, or may be a business that provides distributed images of games and so forth.

Here, the “gift function” provided by game viewers means, for example, a function of sending money or items that can be exchanged for money from a viewer to the distributor in return for support or a high rating in the live distribution of a game video.

Consequently, when it is detected that the game is becoming more exciting on the basis of the level of concentration of a viewer watching the game screen, for example, control can be performed to execute the gift function for the distributor who distributes the video of the game.

The gift function may be executed, for example, when the excitement of the game reaches a specific level or higher, the execution of the gift function is proposed to the viewer, and the viewer finally carries out a payment operation.

The communication control unit 21 performs push communication to prompt game viewers to watch the game screen on the basis of the prediction result from the intense scene prediction unit 19, since an intense scene will be coming up soon.

Consequently, viewers of the game can be prompted not to miss an intense scene by notifying the viewers that the occurrence of an intense scene has been predicted according to pulse wave fluctuations, etc.

Also, the communication control unit 21 performs push communication to the viewers of the game in order to notify the other viewers that there is an intense scene that viewers are concentrating on, on the basis of the estimation result of the level of concentration of the viewers as estimated by the concentration estimation unit 14.

Consequently, the excitement of the game is detected according to the estimated level of concentration of the viewers, and the viewers can be prompted to watch by notifying the viewers of other games about an exciting game scene, according to the degree of excitement.

The storage unit 22 stores facial images including the faces of game players and viewers received from the plurality of cameras 30a and 30b. The storage unit 22 stores, for example, the detection result from the pulse wave detection unit 13, the estimation results from the concentration estimation unit 14 and the emotion estimation unit 15, the evaluation result from the evaluation unit 16, the detection result from the intense scene detection unit 18, the prediction result from the intense scene prediction unit 19, and other such information.

### Display of Game Screen 40 During Play

With the content evaluation device 10 of this embodiment, as shown in FIG. 4, for example, the display control unit 17 displays information such as the level of concentration of each player as estimated by the concentration estimation unit 14 on the game screen 40 during play, in the video of the game actual situation on the live distribution site.

The game screen 40 is, for example, a distribution screen of a game video provided to viewers, and as shown in FIG. 4, a game character (operation target P1) operated by the player is displayed near the center.

As shown in FIG. 4, a facial image display area 41, in which the player's own facial image is displayed, is provided to the upper left of the operation target P1. A level of concentration display area 42, in which changes in the player's level of concentration (estimation result) displayed in the facial image display area 41, is provided near the right side of the facial image display area 41, in a barometer format.

Also, as shown in FIG. 4, the game screen 40 is provided with a facial image display area 43 in which the facial image of a battle opponent (enemy) is provided to the upper right of the game character (operation target P1). A level of concentration display area 44, in which changes in the player's level of concentration (estimation result) displayed in the facial image display area 43, is provided near the right side of the facial image display area 43 in a barometer format.

Furthermore, as shown in FIG. 4, a game participant display area 45, in which are displayed the names of other participants in the game, numerals indicating the number of viewers watching the game screens of those participants, etc., is provided on the left side of the game screen 40.

As shown in FIG. 4, a comment display area 46, in which are displayed comments from viewers watching the displayed game screen 40 during play, is provided on the right side of the game screen 40.

The facial image display area 41 displays the player's own facial image taken by a camera 30a (such as a camera installed in a PC or the like) installed in front of the player operating the game character (operation target P1).

The level of concentration display area 42 displays changes in the level of concentration estimated from the player's facial image, in a barometer format.

The facial image display area 43 displays the facial image of another player, captured by the camera 30a (such as a camera installed in a PC or the like) installed in front of the other player who will be the opponent (enemy) in the battle.

The level of concentration display area 44 displays, in a barometer format, changes in the level of concentration estimated from the facial images of the other player who is an opponent (enemy) in the battle.

The player's level of concentration displayed in the level of concentration display areas 42 and 44 may be estimated using the player's facial image displayed in the facial image display areas 41 and 43 of the game screen 40, or may be estimated using an image acquired from the camera 30a actually installed in front of the player.

The game participant display area 45 displays, for example, the names of other participants (players) participating in a game in which multiple players can participate in a video of the game actual situation on a live distribution site, numerals indicating the number of viewers who are watching the game screens of those participants, etc.

A viewer uses an input device such as a mouse, keyboard, controller, or the like to select a player displayed in this game participant display area 45. Consequently, the game screen 40 to be viewed can be switched so that the player can view the game screen 40 being played.

The comment display area 46 displays, for example, comments posted by viewers who are watching the game screen 40 being played, which are displayed in a video of the game actual situation on a live distribution site, with these comments being regularly updated along with the user names of the posters. The viewer comments displayed in the comment display area 46 are, for example, their expressions of gratitude toward the distributor. The post count increases every time the player is involved in an intense scene, and the rating value indicated by “Total 32,756” in the viewer evaluation area 46a shown in FIG. 4 increases. Also, by analyzing the content of the comments displayed in the comment display area 46, it is possible to estimate how exciting the game is.

Therefore, the excitement level can be ranked and displayed for each community made up of game players and viewers, along with other communities, for example.

Also, the comment display area 46 displays a camera mark 46b indicating that a viewer who has turned on an intense battle sensor (not shown), for example, is being captured by a camera 30b that captures that viewer's own facial image.

Consequently, viewers can enjoy the feeling of having participated in the intense scenes in the game, and can convey to the distributor in real time the excitement experienced by watching the game. Furthermore, in the case of live-streamed game videos, for example, viewers are able to share the excitement of the live experience with the world through an Internet connection, thereby gaining more response.

Here, the detection of intense scenes based on the estimation result for the level of concentration of the plurality of players playing the game and the plurality of viewers watching the game screen 40, the detection of how frequently these scenes occur, the prediction of the occurrence of an intense scene, the gift function according to the excitement level, and the community ranking function are set as functions at the game video distribution site, for example.

Consequently, it is possible to provide the players playing the game and the viewers watching the game screen 40 with additional functions in the video of the game actual situation.

Each function may be set to be a free or paid function depending on its service level.

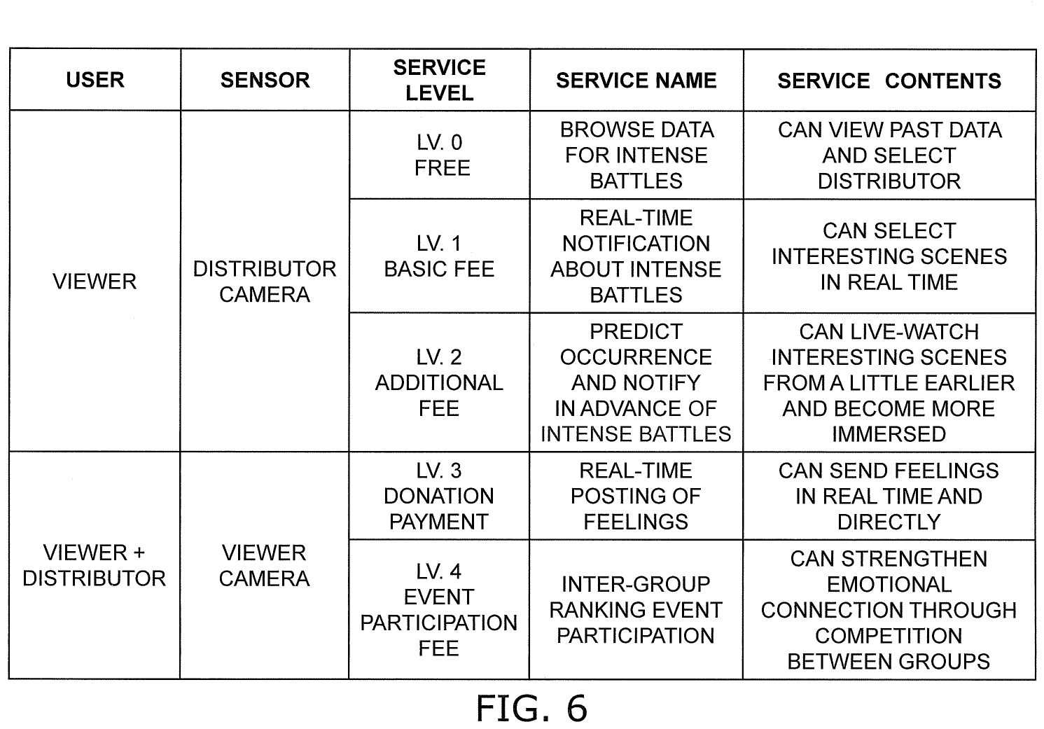

For instance, as shown in FIG. 6, as examples of services for viewers using images captured by the cameras 30a on the player side, data browsing of intense battles (Lv. 0) (free), real-time notification of intense battles (Lv. 1) (basic charge), occurrence prediction and advance notification of intense battles (Lv. 2) (additional charge), etc., are set.

A viewer can look at past data and select a distributor by enabling data browsing of intense battles in Lv. 0 (free).

A viewer can select and view interesting scenes in real time by enabling real-time notification of intense battles in Lv. 1 (basic fee).

A viewer can live-watch interesting scenes from a little earlier and become more immersed in the scene by enabling advance notification and prediction of the occurrence of intense battles in Lv. 2 (additional charge).

Also, as examples of services for viewers and distributors using images captured by the viewer-side cameras 30b, real-time posting of feelings (Lv. 3) (donation payment), inter-group ranking event participation (Lv. 4) (event participation fee), etc., are set.

A viewer can transmit feelings directly and in real time to distributors by enabling real-time posting of feelings in Lv. 3 (donation payment).

By enabling inter-group ranking event participation in Lv. 4 (event participation fee), the emotional connection between viewers and distributors who belong to the same group can be strengthened through competition between groups.

Consequently, by building a system that charges according to the level of feelings, for example, it is possible to build a system that earns profits for the distributor while giving viewers an emotional experience.

### Content Evaluation Method

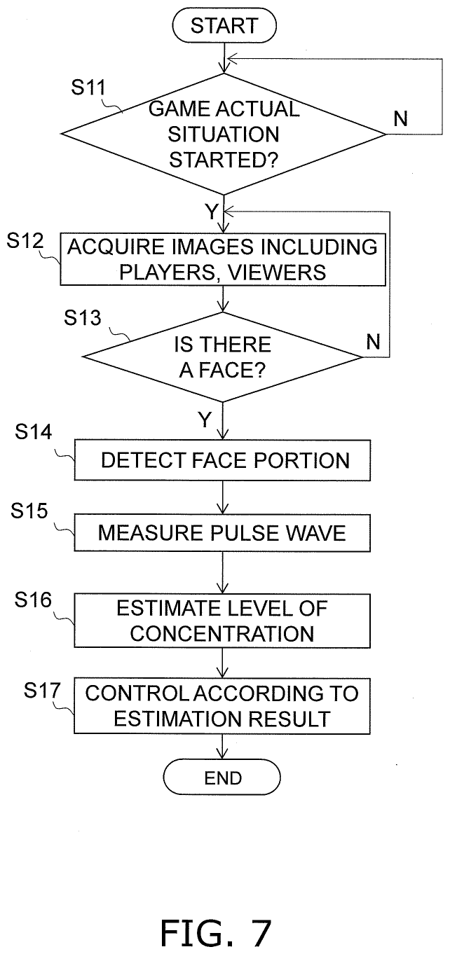

The content evaluation device 10 of this embodiment has the configuration described above, and evaluates content according to the flowcharts shown in FIGS. 7 to 12.

That is, as shown in the main flow of FIG. 7, in step S11 the content evaluation device 10 first determines whether or not a game actual situation has started on the game screen 40 with the game actual situation shown in FIG. 4, for example. If the answer is no, the system waits until a game situation starts, and if the answer is yes, the flow proceeds to step S12.

Next, in step S12, the image acquisition unit 11 acquires from the cameras 30a and 30b the facial images of one or more players participating in the game and the facial images of the viewers watching the live video of the game actual situation.

The images acquired by the image acquisition unit 11 are, as discussed above, images of the subject that are continuously captured in time series in order to detect the pulse waves of the players and the viewers.

Next, in step S13, it is determined whether or not an acquired image includes a portion where the skin is exposed (such as the face). If such a portion is included, the processing proceeds to step S14.

Next, in step S14, the detection area setting unit 12 sets the face portion included in the image as a detection area.

Next, in step S15, the pulse wave detection unit 13 calculates the average luminance value of the pixels included in the detection area for the face portion set in step S14, and analyzes the change over time to detect the pulse waves of the players and the viewers.

Next, in step S16, the concentration estimation unit 14 estimates the levels of concentration of the players and the viewers on the basis of the pulse waves detected in step S15.

In the estimation of the level of concentration in step S16, in addition to the detection result from the pulse wave detection unit 13, the concentration estimation unit 14 may estimate the level of concentration on the basis of the estimation result from the emotion estimation unit 15 (joy, surprise, fear, anger, disgust, sadness, etc.), for example.

Next, in step S17, various controls (discussed below) are performed according to the levels of concentration of the game players and viewers estimated in step S16.

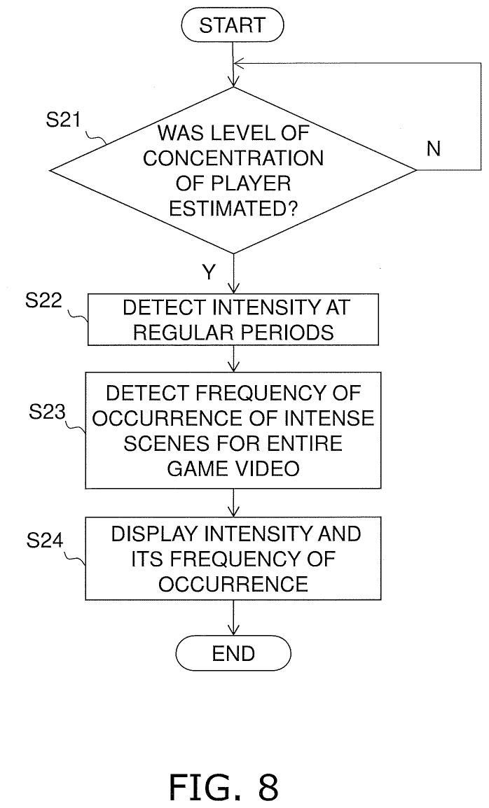

Here, the “control of the estimation result” in step S17 will be described below in specific terms using the flowcharts shown in FIGS. 8 to 12.

### Control 1 According to Player's Level of Concentration

As shown in FIG. 8, in step S21, it is determined whether or not the level of concentration estimated in step S16 is that of a game player. Here, if this is the level of concentration of a player, the processing proceeds to step S22, but if it is the level of concentration of a viewer, the system waits until the level of concentration of a player is estimated.

Next, in step S22, the intense scene detection unit 18 detects the intensity at specific time intervals on the basis of the estimated increase or decrease in the level of concentration of the player.

Here, the intensity means, for example, the degree to which the game player's excitement rises in the above-mentioned intense scene.

In step S22, instead of the increase or decrease in the level of concentration, the intensity may be detected at specific time intervals on the basis of the frequency with which the estimated level of concentration of the player exceeds a specific threshold.

Next, in step S23, the intense scene detection unit 18 detects the frequency at which intense scenes occur in the entire game video on the basis of the estimated increase or decrease in the level of concentration of the player.

Next, in step S24, the display control unit 17 causes the game screen 40 to display the intensity detected in steps S22 and S23 and the frequency of occurrence of intense scenes, over an Internet line, for example.

Consequently, in the flowchart showing step S6, the evaluation unit 16 can evaluate the content of the game by detecting the intensity and the frequency at which intense scenes occur, as the degree of excitement of the game.

### Control 2 According to Player's Level of Concentration

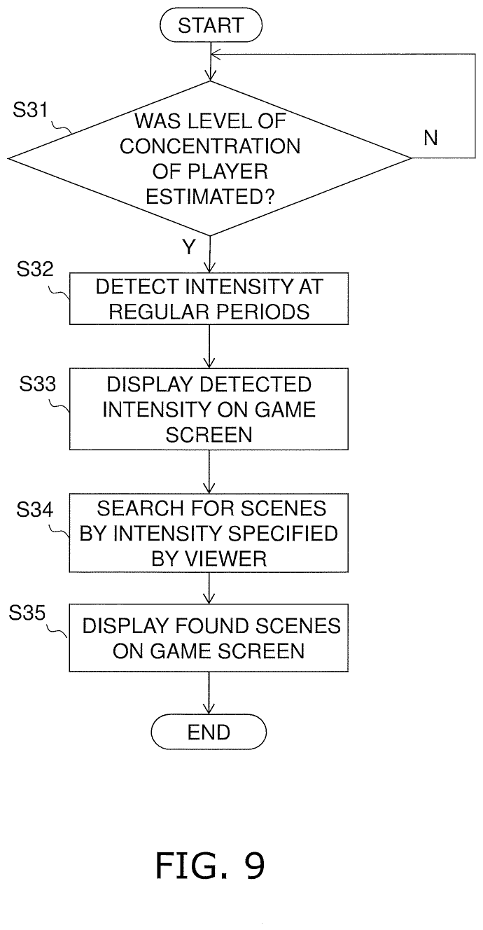

As shown in FIG. 9, in step S31, it is determined whether or not the level of concentration estimated in step S16 is that of a game player. Here, if it is the level of concentration of a player, the processing proceeds to step S32, but if it is the level of concentration of a viewer, the system waits until the level of concentration of a player is estimated.

Next, in step S32, just as in step S22, the intense scene detection unit 18 detects the intensity at specific time intervals on the basis of the estimated increase or decrease in the player's level of concentration.

Next, in step S33, the display control unit 17 causes the game screen 40 to display the intensity detected in step S32, over an Internet line, for example.

Next, in step S34, a viewer who is watching the game screen 40 on which the intensity is displayed searches for the intensity that that viewer wants to see (such as the scene with the highest intensity).

Next, in step S35, the display control unit 17 causes the game screen 40 to display a scene corresponding to the intensity searched for in step S34, over an Internet line, for example.

This allows the viewer to select and view the intense scenes of the game that are detected according to the level of concentration of the player.

### Control 3 According to Player's Level of Concentration

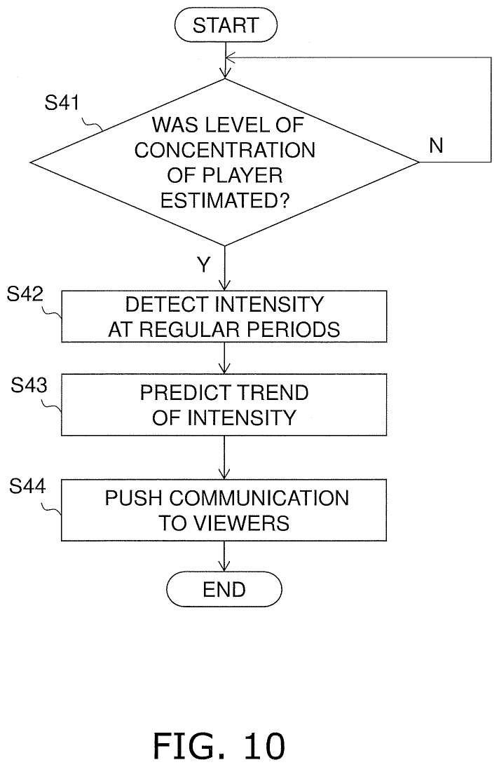

As shown in FIG. 10, in step S41, it is determined whether or not the level of concentration estimated in step S16 is that of a game player. Here, if it is the level of concentration of a player, the processing proceeds to step S42, but if it is the level of concentration of a viewer, the system waits until the level of concentration of a player is estimated.

Next, in step S42, just as in steps S22 and S32, the intense scene detection unit 18 detects the intensity at specific time intervals on the basis of the estimated increase or decrease in the player's level of concentration.

Next, in step S43, the intense scene prediction unit 19 predicts the trend of intensity, such as the relation between the increase or decrease in the level of concentration of the player and the increase or decrease in the intensity.

Next, in step S44, the communication control unit 21 performs push communication to the viewers viewing other screens 40, sending, for example, a message prompting those viewers to look at the game screen 40 being played by a player in a state in which the intense scene predicted in step S43 is predicted to occur.

Consequently, viewers who are currently watching other players' game screens, or viewers who are currently watching other content, for example, can be notified in advance before a intense scene occurs, so the viewers will not miss the scenes they want to watch.

Therefore, the evaluation of the community to which the viewers belong can be improved by increasing the number of viewers who can view intense scenes without missing them, for example.

### Control 1 According to Viewer's Level of Concentration

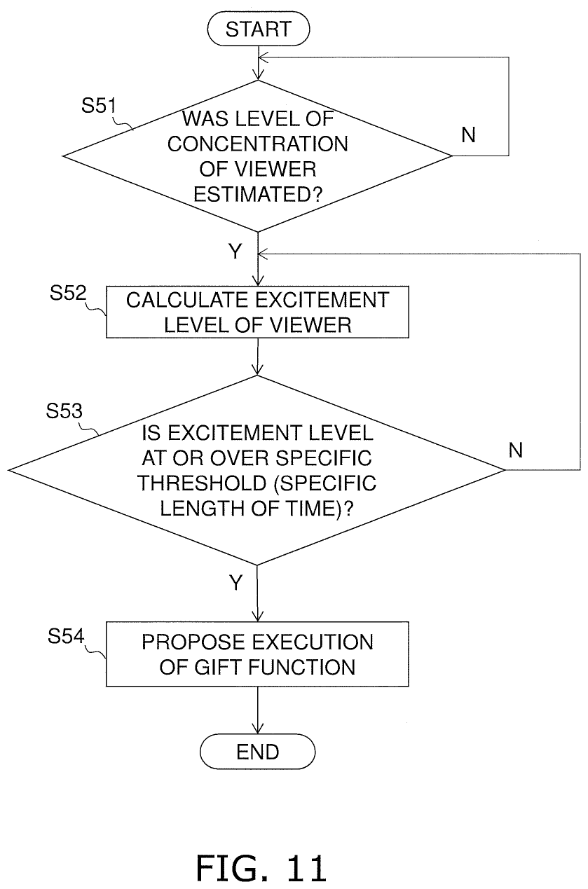

As shown in FIG. 11, in step S51, it is determined whether or not the level of concentration estimated in step S16 is that of a viewer viewing the game screen 40. Here, if it is a viewer's level of concentration, the processing proceeds to step S52, but if it is a player's level of concentration, the system waits until a viewer's level of concentration is estimated.

Next, in step S52, the intense scene detection unit 18 detects the excitement level at specific points in time on the basis of the estimated increase or decrease in the level of concentration of the viewers.

Next, in step S53, the intense scene detection unit 18 determines whether or not the viewer excitement level calculated in step S52 is at or above a specific threshold value, or whether or not it is at or above a specific length of time.

Here, if it is at or above the specific threshold value or the specific length of time, the processing proceeds to step S54, but if it is below the specific threshold value or the specific length of time, the processing returns to step S52 and the excitement level of the viewers is calculated again.

Next, in step S54, since it was determined in step S53 that the viewer excitement level calculated in step S52 was at or above the specific threshold value or the specific length of time, the gift control unit 20 proposes the execution of a gift (such as a tip) function to the distributor who distributes to the viewers.

Consequently, when the excitement level of the viewers is at or above a specific threshold value or has lasted for at least a specific length of time, the viewers are automatically prompted to execute the gift function, which makes it easy to execute a gift function according to the excitement level.

### Control 2 According to Viewer's Level of Concentration

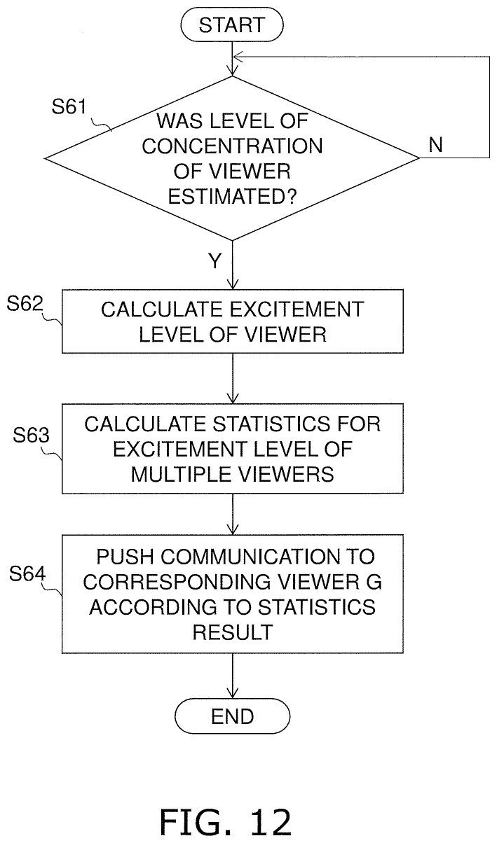

As shown in FIG. 12, in step S61, it is determined whether or not the level of concentration estimated in step S16 is that of a viewer viewing the game screen 40. Here, if it is a viewer's level of concentration, the processing proceeds to step S62, but if it is a player's level of concentration, the system waits until a viewer's level of concentration is estimated.

Next, in step S62, just as in step S52, the intense scene detection unit 18 detects the excitement level at specific points in time on the basis of the estimated increase or decrease in the level of concentration of the viewers.

Next, in step S63, the intense scene detection unit 18 calculates statistics for the excitement level of the viewers.

Next, in step S64, the communication control unit 21 performs push communication of information that informs the group to which a plurality of viewers belong about how exciting a game (content) is, on the basis of the statistics about the excitement level of the viewers calculated in step S63.

Consequently, information about game videos that other viewers are excited about is provided to the viewers who are currently watching other players' game screens, or viewers who are currently watching other content, etc., which allows the viewers to select and watch the game videos they want to see.

### Other Embodiments

An embodiment of the present invention was described above, but the present invention is not limited to or by the above embodiment, and various modifications are possible without departing from the gist of the invention.

### (A)

In the above embodiment, an example was given in which the present invention was realized as the content evaluation device (pulse wave detection device) 10 and a content evaluation method (pulse wave detection method). However, the present invention is not limited to this.

For example, the present invention may be realized as a pulse wave detection program that causes a computer to execute a pulse wave detection method featuring a pulse wave detection device.

This pulse wave detection program is stored in a memory (storage unit) installed in the pulse wave detection device, and the CPU reads the pulse wave detection program stored in the memory and causes the hardware to execute the various steps. More specifically, the CPU reads the pulse wave detection program and executes the above-mentioned image acquisition step, detection area setting step, and pulse wave detection step, thereby obtaining the same effect as described above.

Also, the present invention may be realized as a recording medium on which is stored a pulse wave detection program for a pulse wave detection device.

### (B)

In the above embodiment, an example was given in which the game to be evaluated was a battle-type game in which a plurality of players can participate simultaneously. However, the present invention is not limited to this.

For example, a game that is played alone may be evaluated instead of a battle-type game.

### (C)

In the above embodiment, an example was given in which the pulse waves of both players and viewers of the game were detected, and the game was evaluated on the basis of the results of estimating the levels of concentration of each person. However, the present invention is not limited to this.

For example, the game may be evaluated by using the pulse wave detection results and concentration estimation results for just the game players.

Alternatively, the game may be evaluated by using the pulse wave detection results and the level of concentration estimation results for just the viewers who are watching the game screen.

### (D)

In the above embodiment, an example was given in which the game was evaluated according to the level of concentration of each player, as estimated on the basis of the pulse wave detection results for the game players and viewers. However, the present invention is not limited to this.

For example, the detected pulse waves may be used to detect the state of the game players or viewers, in addition to being used to estimate the level of concentration.

Also, the level of concentration estimated on the basis of the detected pulse waves may be used for purposes other than evaluating the game.

### (E)

In the above embodiment, an example was given in which the game was evaluated by taking into account the emotions of the players and viewers estimated from the facial images of the players and viewers, in addition to the estimation results for level of concentration based on the pulse waves of the game players and viewers. However, the present invention is not limited to this.

For example, the game may be evaluated by using only the estimation results for the level of concentration of each person based on the pulse waves, without using the estimation results for the emotions of the players and the viewers.

### (F)

In the above embodiment, an example was given in which the pulse waves of each person were detected using facial images of the game players and viewers. However, the present invention is not limited to this.

For example, a body part other than the face, such as the arms of the players or viewers during game play, may be set as a detection area to detect pulse waves.

### (G)

In the above embodiment, an example was given in which the game was evaluated in real time by using images of the players and viewers during game play. However, the present invention is not limited to this.

For example, the game may be evaluated after the fact, using a recorded video or the like.

### (H)

In the above embodiment, an example was given in which facial images were acquired from video including game players and viewers captured by the plurality of cameras 30a and 30b, and the pulse waves of each person were detected and the level of concentration was estimated. However, the present invention is not limited to this.

For example, a video stored on a server or the like may be downloaded to detect the pulse waves of the game players and/or viewers and to estimate the level of concentration.

### (I)

In the above embodiment, an example was given in which the player's estimated level of concentration was displayed on the screen during the game. However, the present invention is not limited to this.

For example, the estimated level of concentration may not be displayed on the game screen, and instead the detected pulse waves may be displayed on the game screen.

### (J)

In the above embodiment, an example was given in which the game was evaluated by using a plurality of levels of concentration estimated from the pulse waves of a plurality of game players and a plurality of viewers. However, the present invention is not limited to this.

For example, the game may be evaluated on the basis of a user's level of concentration estimated from the pulse waves of a single game player or viewer.

### (K)

In the above embodiment, an example was given in which the content evaluation device 10 acquired facial images of game players and viewers from a plurality of cameras 30a and 30b provided outside the device. However, the present invention is not limited to this.

For example, if the configuration is such that the pulse wave detection device is integrated with a camera, then the camera itself will function as an image acquisition unit, and the same effect as that obtained with the present invention can be obtained in the same manner as in the above embodiment.

### (L)

In the above embodiment, an example was given in which the plurality of cameras 30a and 30b were provided as the means for capturing facial images of game players and viewers. However, the present invention is not limited to this.

For example, if there is only one player and/or viewer of the game and one image is acquired, the configuration may be such that an image of the player, etc., is captured by a single camera.

### INDUSTRIAL APPLICABILITY

The pulse wave detection device of the present invention exhibits the effect of being able to detect the pulse waves of players or the like during game play in a non-contact manner, and thereby estimate the state of the player or the like with respect to the game, and as such can be widely applied to devices, systems, and so forth that perform evaluations of various kinds of game, etc.

### REFERENCE SIGNS LIST

10 content evaluation device (pulse wave detection device)
11 image acquisition unit
12 detection area setting unit
13 pulse wave detection unit
14 concentration estimation unit
15 emotion estimation unit
16 evaluation unit
17 display control unit
18 intense scene detector
19 intense scene predictor
20 gift control unit
21 communication control unit
22 storage unit
30a camera
30b camera
40 game screen
41 facial image display area
42 level of concentration display area
43 facial image display area
44 level of concentration display area
45 game participant display area
46 comment display area
46a viewer evaluation area
46b camera mark
50 content evaluation system
P1 operation target

1. A pulse wave detection device that detects the pulse wave of a player playing a game, the pulse wave detection device comprising:
an image acquisition unit configured to acquire an image including the player;
a detection area setting unit configured to set a detection area including a skin of the player included in the image acquired by the image acquisition unit; and
a pulse wave detection unit configured to detect the player's pulse wave from the detection area set by the detection area setting unit.

2. The pulse wave detection device according to claim 1,
further comprising a concentration estimation unit configured to estimate a level of concentration of the player with respect to the game according to a detection result of the pulse wave of the player.

3. The pulse wave detection device according to claim 2,
further comprising an evaluation unit configured to evaluate the game on the basis of an estimation result of the concentration estimation unit.

4. The pulse wave detection device according to claim 3,
wherein the evaluation unit evaluates the game according to the estimation result for the level of concentration of a plurality of the players.

5. The pulse wave detection device according to claim 3,
wherein the evaluation unit evaluates the game when a state in which the level of concentration by the player is at or over a specific threshold has lasted for at least a specific number of times or for at least a specific length of time.

6. The pulse wave detection device according to claim 2,
further comprising a display control unit configured to cause an estimation result of a concentration estimation unit to be displayed on a display screen of the game.

7. The pulse wave detection device according to claim 2,
further comprising an intense scene detection unit configured to detect an exciting scene of the game on the basis of the level of concentration of a plurality of the players with respect to the game, estimated by the concentration estimation unit according to the detection result of the pulse wave detection unit.

8. The pulse wave detection device according to claim 7,
further comprising an intense scene prediction unit configured to predict the intense scene on the basis of an estimation result of the concentration estimation unit.

9. The pulse wave detection device according to claim 8,
further comprising a communication control unit configured to perform push communication to a viewer of the game on the basis of the basis of a prediction result of the intense scene prediction unit.

10. The pulse wave detection device according to claim 2,
wherein the concentration estimation unit estimates the level of concentration for each player by taking into account a difficulty level of the game and an ability of each player.

11. The pulse wave detection device according to claim 2,
wherein the image acquisition unit acquires an image of a viewer who is viewing a screen of the game, and
the concentration estimation unit uses an image of the viewer to estimate the level of concentration of the viewer.

12. The pulse wave detection device according to claim 11,
further comprising a gift control unit configured to control execution of a gift function from the viewer to a distributor who is distributing a video of the game, on the basis of an estimation result for the level of concentration of the viewer estimated by the concentration estimation unit.

13. The pulse wave detection device according to claim 11,
further comprising an evaluation unit configured to evaluate the game on the basis of an estimation result for the concentration estimation unit,
wherein the evaluation unit evaluates the game on the basis of the level of concentration of both the player and the viewer.

14. The pulse wave detection device according to claim 11,
further comprising a communication control unit configured to perform push communication to the viewer of the game on the basis of an estimation result for the level of concentration of the viewer estimated by the concentration estimation unit.

15. The pulse wave detection device according to claim 1,
further comprising an emotion estimation unit configured to estimate a player's emotion on the basis of information about a facial portion of the player set in the detection area setting unit.

16. A pulse wave detection method for detecting a pulse wave of a player while playing a game, the method comprising:
an image acquisition step of acquiring an image including the player;
a detection area setting step of setting a detection area including a skin of the player included in the image acquired in the image acquisition step; and
a pulse wave detection step of detecting the player's pulse wave from the detection area set in the detection area setting step.

17. A pulse wave detection program for detecting a pulse wave of a player during game play, the program causing a computer to execute a pulse wave detection method, the method comprising:
an image acquisition step of acquiring an image including the player;
a detection area setting step of setting a detection area including a skin of the player included in the image acquired in the image acquisition step; and
a pulse wave detection step of detecting the player's pulse wave from the detection area set in the detection area setting step.

18. A pulse wave detection device, comprising:
an image acquisition unit configured to acquire an image including a viewer who is watching a screen of the game during game play;
a detection area setting unit configured to set a detection area including a skin of the viewer included in the image acquired by the image acquisition unit; and
a pulse wave detection unit configured to detect the pulse wave of the viewer from the detection area set in the detection area setting unit.

19. The pulse wave detection device according to claim 18,
further comprising a concentration estimation unit configured to estimate a level of concentration of the viewer with respect to the game according to a detection result of the pulse wave of the viewer.

20. The pulse wave detection device according to claim 19,
further comprising an evaluation unit configured to evaluate the game on the basis of an estimation result of the concentration estimation unit.

21. The pulse wave detection device according to claim 20,
wherein the evaluation unit evaluates the game according to the estimation result for the level of concentration of a plurality of the viewers.

22. The pulse wave detection device according to claim 20,
wherein the evaluation unit evaluates the game when a state in which the level of concentration by the viewer is at or over a specific threshold has lasted for at least a specific number of times or for at least a specific length of time.

23. The pulse wave detection device according to claim 19,
further comprising a gift control unit configured to control execution of a gift function from the viewer to a distributor who is distributing a video of the game, on the basis of an estimation result for the level of concentration of the viewer estimated by the concentration estimation unit.

24. The pulse wave detection device according to claim 19,
further comprising a communication control unit configured to perform push communication to the viewer of the game on the basis of an estimation result for the level of concentration of the viewer estimated by the concentration estimation unit.

25. The pulse wave detection device according to claim 19,
further comprising an intense scene detection unit configured to detect an exciting scene of the game on the basis of the level of concentration of a plurality of the viewers with respect to the game, estimated by the concentration estimation unit according to the detection result of the pulse wave detection unit.

26. The pulse wave detection device according to claim 25,
further comprising an intense scene prediction unit configured to predict the intense scene on the basis of an estimation result of the concentration estimation unit.

27. The pulse wave detection device according to claim 26,
further comprising a communication control unit configured to perform push communication to a viewer of the game on the basis of a prediction result of the intense scene prediction unit.

28. The pulse wave detection device according to claim 19,
wherein the image acquisition unit acquires an image of a player playing the game, and
the concentration estimation unit uses an image of the player to estimate the level of concentration of the player.

29. The pulse wave detection device according to claim 28,
further comprising an evaluation unit configured to evaluate the game on the basis of an estimation result of the concentration estimation unit,
wherein the evaluation unit evaluates the game on the basis of the level of concentration of both the viewer and the player.

30. The pulse wave detection device according to claim 18,
further comprising an emotion estimation unit configured to estimate a viewer's emotion on the basis of information about a facial portion of the viewer set in the detection area setting unit.

31. A pulse wave detection method, comprising:
an image acquisition step of acquiring an image including a viewer who is viewing a game screen during game play;
a detection area setting step of setting a detection area including a skin of the viewer included in the image acquired in the image acquisition step; and
a pulse wave detection step of detecting the viewer's pulse wave from the detection area set in the detection area setting step.

32. A pulse wave detection program causing a computer to execute a pulse wave detection method, the method comprising:
an image acquisition step of acquiring an image including a viewer who is viewing a game screen during game play;
a detection area setting step of setting a detection area including a skin of the viewer included in the image acquired in the image acquisition step; and
a pulse wave detection step of detecting the viewer's pulse wave from the detection area set in the detection area setting step.
